(19) United States
(12) Patent Application Publication
(10) Pub. No.: US 2012/0100071 A1
(43) Pub. Date: Apr. 26, 2012

(54) SYNTHESIS AND USE OF RADIOLABELLED INSULIN ANALOGUES

(76) Inventors: John Valliant, Ancaster (CA); Chitra Sundararajan, Hamilton (CA); Katharina Guenther, Ottawa (CA); Travis Besanger, Waterdown (CA)

(21) Appl. No.: 13/375,729

(22) PCT Filed: Jun. 7, 2010
(86) PCT No.: PCT/CA2010/000855

§ 371 Date: Jan. 9, 2012

Related U.S. Application Data
(60) Provisional application No. 61/184,481, filed on Jun. 5, 2009.

(51) Int. Cl. A61K 51/08; A61P 5/48; C07K 14/62

(52) U.S. Cl. 424/1.69; 530/303; 530/304

(57) ABSTRACT

A radiolabelled insulin analogue is provided. The analogue comprises a radiolabel linked to an insulin analogue at an amino acid at the terminal end of the B chain of the insulin analogue.

### FIELD OF THE INVENTION

The present invention relates to novel molecular imaging probes that are suitable for use in vivo.

### BACKGROUND OF THE INVENTION

Insulin is a polypeptide-based hormone that is featured prominently in energy homeostasis through its regulation of glucose uptake and influence over energy storing metabolites including lipids and proteins. As a result of its central role in energy metabolism, abnormalities in insulin regulation are associated with a variety of diseases including diabetes, hypertension, and cancer. Molecular imaging agents derived from human insulin for use in radio-imaging studies offer a valuable, non-invasive means to study diseases that involve insulin disregulation in vivo. A number of radiolabelled insulin analogues have been reported, including ¹²⁵I-insulin, which is widely used for in vitro insulin receptor (IR) binding assays, and ¹²⁴I-insulin and ¹⁸F-insulin bioconjugates for positron emission tomography (PET) studies.

Technetium-99m remains the most widely used isotope in nuclear medicine due to its low cost, widespread availability, and attractive nuclear properties (E_{γ}=140 keV, t_{1/2}=6.02 h). As a result, a ^{99m}Tc-insulin analogue is particularly attractive for use as a single photon emission computed tomography (SPECT) probe of insulin biochemistry. ^{99m}Tc-labeled insulin has previously been prepared by treating the hormone directly with ^{99m}TcO₄⁻ in the presence of SnCl₂. The labeling strategy resulted in reduction of the disulfide bonds and the formation of multiple labeled products, which limited the ability of the approach to generate a true insulin mimic.

In view of the foregoing, it would be desirable to develop alternative molecular imaging probes.

### SUMMARY OF THE INVENTION

A novel radiolabelled insulin analogue has now been prepared using a method developed to label insulin in a regioselective manner at a site on the hormone that was designed to minimize the impact of the radiometal.

Accordingly, in one aspect of the invention, a radiolabelled insulin analogue is provided comprising a radiolabel linked to an insulin analogue at an amino acid at the terminal end of the B chain of the insulin analogue.

In another aspect of the invention, a method of preparing a radiolabelled insulin analogue is provided comprising the steps of:
i) linking a chelator to a terminal amino group on the B-chain of the insulin analogue; and
ii) reacting the linked chelator with a radioisotope under suitable conditions.

In a further aspect of the invention, a kit is provided comprising a chelator-linked insulin analogue and a radioisotope to be reacted therewith.

These and other aspects of the invention will become apparent in the detailed description by reference to the following figures.

### BRIEF DESCRIPTION OF THE DRAWINGS

FIG. 1 is a schematic of the synthesis of DBI (1) and AHx-DBI (2) referred to herein as Scheme 1;

FIG. 2 is a schematic of the synthesis of active ester (5) referred to herein as Scheme 2;

FIG. 3 is a schematic of the synthesis of Re-BP-Pen-AHx-Insulin (6) referred to herein as Scheme 3;

FIG. 4 is a schematic of an alternate synthesis of Re-BP-Pen-AHx-Insulin (6) referred to herein as Scheme 4;

FIG. 5 is a schematic of the synthesis of BP-Pen-AHx-DBI (9) referred to herein as Scheme 5;

FIG. 6 is a schematic of an alternate route for the synthesis of BP-Pen-AHx-DBI (9);

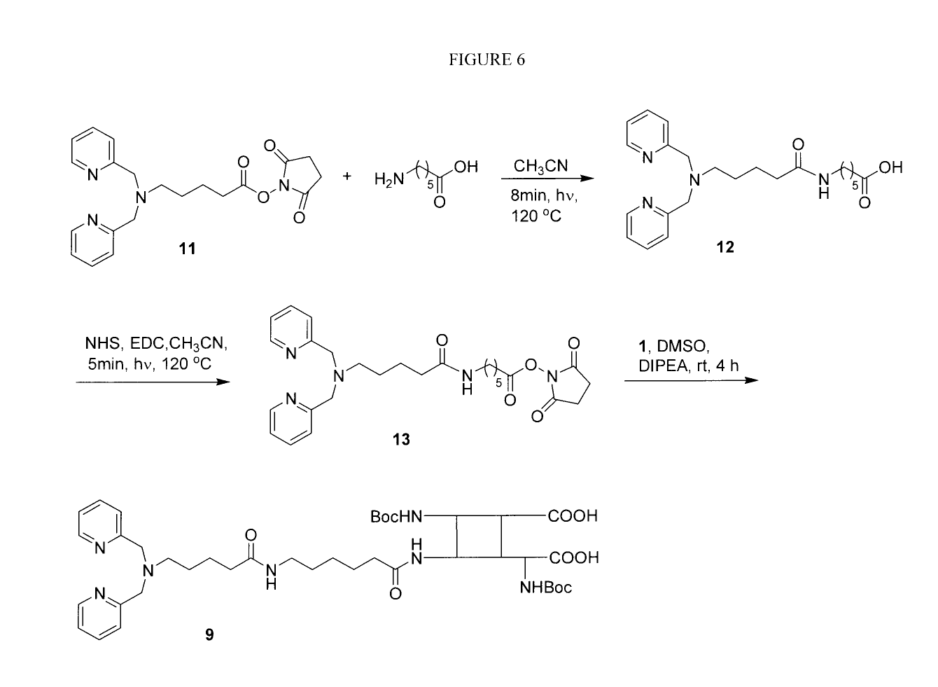

FIG. 7 is a schematic of the synthesis of ^{99m}Tc-BP-Pen-AHx-Insulin (10);

FIG. 8 illustrates human insulin and the target Tc/Re-insulin conjugates;

FIG. 9 illustrates HPLC chromatograms (a) LC-UV obtained using a diode array and monitoring at 310 nm, and (b) LC-MS ESI+ total ion chromatogram scanned from m/z 0-2400, each of compound 6;

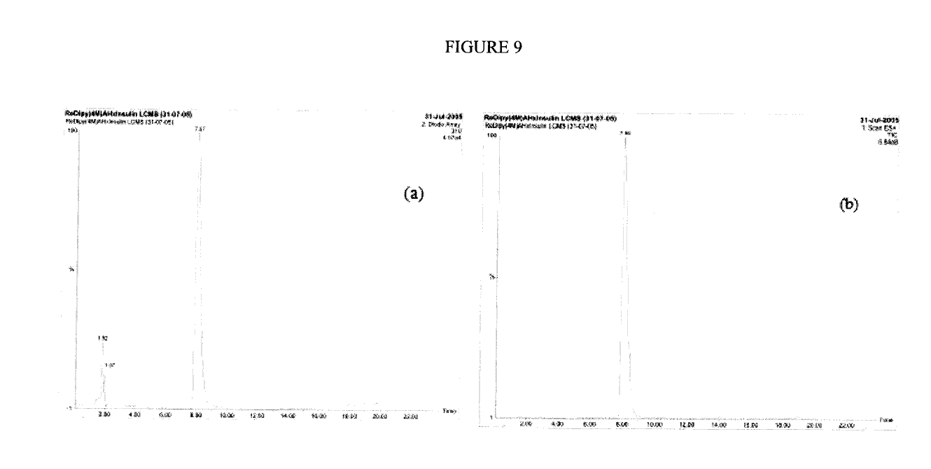

FIG. 10 graphically illustrates a comparison of insulin and compound 6 in vitro using a displacement assay (a), insulin receptor autophosphorylation ELISA (b) and Akt phosphorylation ELISA (c); and

FIG. 11 illustrates radio HPLC chromatograms of (a) crude reaction mixture following the reaction of [^{99m}Tc(CO₃)(OH₂)₃]⁺ with 9 (b) crude reaction mixture following deprotection of ^{99m}Tc-BP-Pen-Ahx-DBI with TFA and anisole and (c) purified 10.

### DETAILED DESCRIPTION OF THE INVENTION

A novel radiolabelled insulin analogue is provided comprising a radiolabel linked to an insulin analogue at an amino acid at a terminal end of the B chain of the insulin analogue.

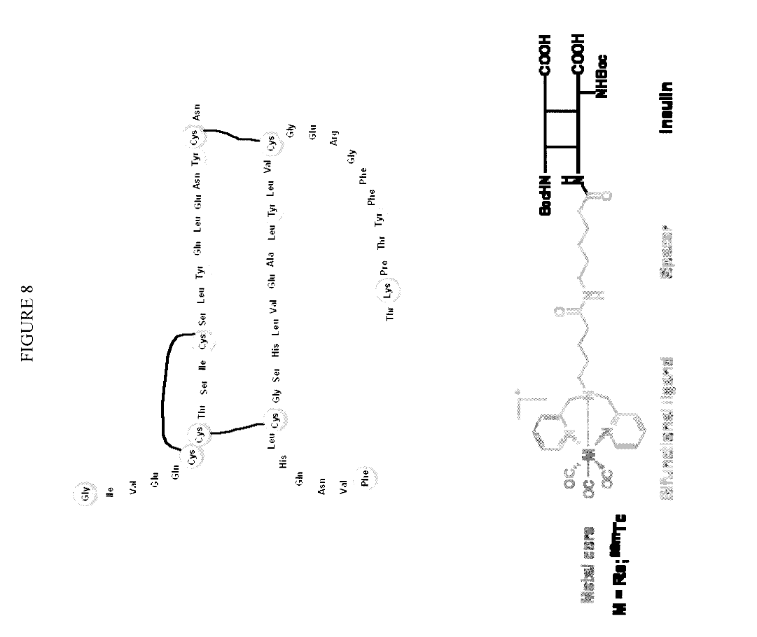

The term insulin analogue is used herein to refer to naturally occurring forms of insulin including human insulin (as shown in FIG. 8), and insulin from other species, including other mammals e.g. porcine and bovine insulin, and from non-mammalian species, e.g. fish. Also encompassed by the term “insulin analogues” are synthetic forms of insulin including recombinant forms of insulin and functionally equivalent modified forms of insulin, e.g. analogues of insulin which include one or more amino acid substitutions, additions or deletions (including but not limited to the reversal of penultimate lysine and proline residues on the C-terminal end of the B-chain; substitution of proline at position 28 on the B chain with aspartic acid; and addition of two arginine residues to the B-chain C-terminus and substitution of asparagine at position 21 with glycine), or analogues which incorporate one or more non-naturally occurring amino acids, or amino acids which have been modified at a functional group thereof. The term “functionally equivalent” refers to an insulin analogue that retains a significant level of activity, e.g. at least about 50% of the activity of native insulin.

The term “radiolabel” or “radioisotope” is used herein to encompass any radioactive isotope suitable for use in vivo, including but not limited to, fluorine-18, gallium-67, krypton-81m, rubidium-82, technetium-99, indium-111, iodine-123, xenon-133 and thallium-201.

The present invention also provides a method of making radiolabelled insulin analogues. The method comprises linking a chelator to a terminal amino group on the B-chain of the insulin analogue. The terminal amino group is preferably an amino group within the last five residues of the N-terminus of the B-chain. Preferably, the terminal amino group is the N-terminus of the B-chain.

Chelators for use in preparing the present analogues include those described in Stephenson et al. in J. Am. Chem. Soc. 2004, 126, 8598-8599 and in Bioconjugate Chem. 2004, 15, 128-136, the contents of which are incorporated herein by reference, including, for example, chelators including a Bis(2-pyridylmethyl) group such as Bis(2-pyridylmethyl) group pentanoic acid. The chelator is linked to the analogue via a spacer of appropriate length, e.g. at least about 3-5 carbon atoms in length, and preferably of greater length, as one of skill in the art will appreciate. The chelator is linked to the insulin analogue via a covalent linkage, such as a peptide linkage.

The chelator-linked analogue is then reacted with a selected radioisotope under suitable conditions. The conditions are selected to minimize non-specific labeling. Appropriate conditions included a pH in the range of about 6-6.8, e.g. about 6.5, a temperature in the range of about 40-50° C., e.g. about 45° C. and a reaction time of at least about 60 minutes, preferably at least about 75 minutes, and more preferably about 90 minutes.

The present radiolabelled insulin analogues are useful to image mammals in the diagnosis of insulin-related disorders, such as diabetes. The method includes administering a radiolabelled insulin analogue to the mammal in a diagnostic amount.

A kit is provided in another aspect of the invention. The kit comprises a chelator-linked insulin analogue as described herein along with a radioisotope to be reacted therewith.

Embodiments of the invention are described by reference to the following specific examples which is not to be construed as limiting.

### Example 1

### Synthesis of a Radiolabelled Insulin Analogue

Synthesis and Characterization of B¹-[Re(CO)₃{bis(2-pyridylmethyl)pentanoyl-6-aminohexanoyl)}]insulin (Re-BP-Pen-AHx-Insulin, 6):

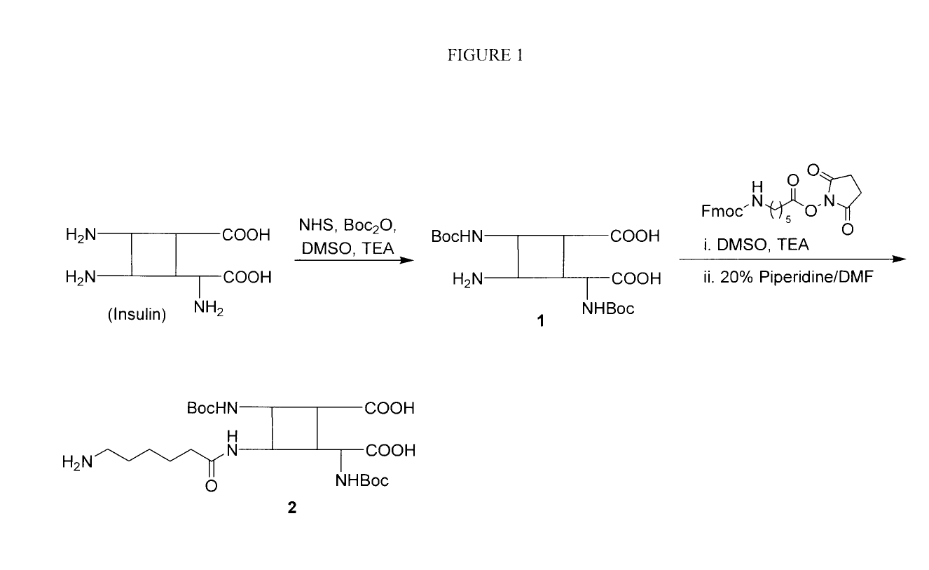

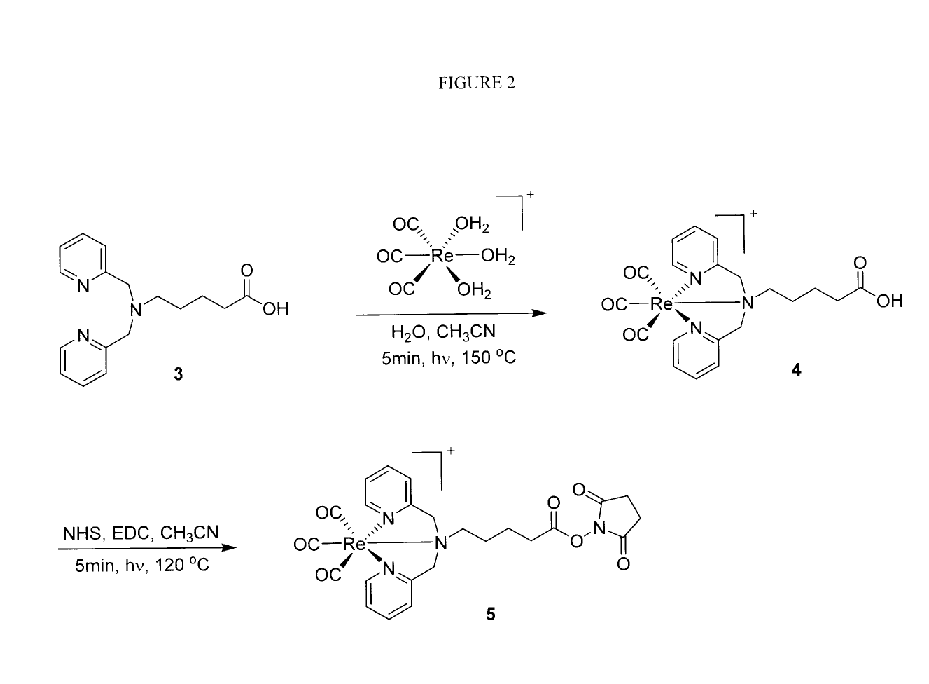

The first step in making a viable ^{99m}Tc-insulin analogue was to isolate the Re analogue of the target and determine if the chosen synthetic route, site of conjugation and nature of the linker group and chelate resulted in any significant alteration of the biochemical properties of the hormone. Re was used as a surrogate since there are no stable isotopes of technetium, and this is a widely accepted approach as the metals are congeners and therefore form isostructural products, particularly in the oxidation state used here. The synthetic route chosen paralleled that previously reported by Shai et al. (Biochem. 1989, 28, 4801-4806) and Guenther et al. (J. Med. Chem. 2006, 49, 1466-1474) in which insulin was modified at the amino-terminus of the PheB1 amino acid residue. This method takes advantage of the differing reactivity of the three primary amines present within the structure of insulin, where the more reactive amines LysB29 and GlyA1 can be protected with Boc groups, leaving the PheB1 amine free for further derivatization. The addition of a short aminohexanoic acid spacer at the PheB1 site was beneficial for labeling with the radionuclide. The initial step towards synthesizing the target compound was to convert insulin to the diBoc derivative 1, followed by conjugation to the aminohexanoic acid spacer via its active ester, to give 2 (FIG. 1). To avoid labeling at reactive amino acid residues and to ensure robust and covalent linkage of the metal to the targeting vector, a bifunctional chelate was employed. The selected chelate (as described by Banerjee et al. Inorg. Chem. 2002, 41, 6417-6425) consisted of the bispyridyl chelate (FIG. 8) that forms a highly stable complex with the M(CO₃)⁺ cores (M=Re, ^{99m}Tc). Succinimidyl [Re(CO)₃(bis(2-pyridylmethyl)pentanoate)] was used to prepare the rhenium standard as it is the metal analogue of succinimidyl-4-fluorobenzoate. The rhenium complex, Re(CO)₃(bis(2-pyridylmethyl)pentanoic acid) 4, was prepared by combining bis(2-pyridylmethyl)pentanoic acid 3 with 1.6 equivalents of [(Re(CO)₃(OH₂)₃]Br in water, and heating the mixture in the microwave at 150° C. for 5 minutes (FIG. 2). The desired product was isolated by silica gel chromatography in 62% yield and its characterization data matches the reported literature data.

To link the Re-complex with the amino group on insulin, a succinimidyl-ester of 4 was generated by mixing 4 and five equivalents each of N-hydroxysuccinimide (NHS) and N-(3-dimethylaminopropyl)-N′-ethylcarbodiimide (EDC) in acetonitrile, and heating at 120° C. for five minutes in a sealed microwave vial. The reaction mixture was evaporated, dissolved in CH₂Cl₂ and washed with water to remove unreacted EDC and EDC-urea byproducts. The resultant mixture was then purified by silica gel chromatography to give the final product, 5, in 91% yield where the corresponding characterization data matched that reported in the literature data. The active ester should be used immediately upon isolation as it rapidly hydrolyses which was confirmed by observing changes in the ¹H NMR over time. In addition, samples of the active esters reported here were difficult to get completely dry and were often isolated with trace amounts of residual solvent.

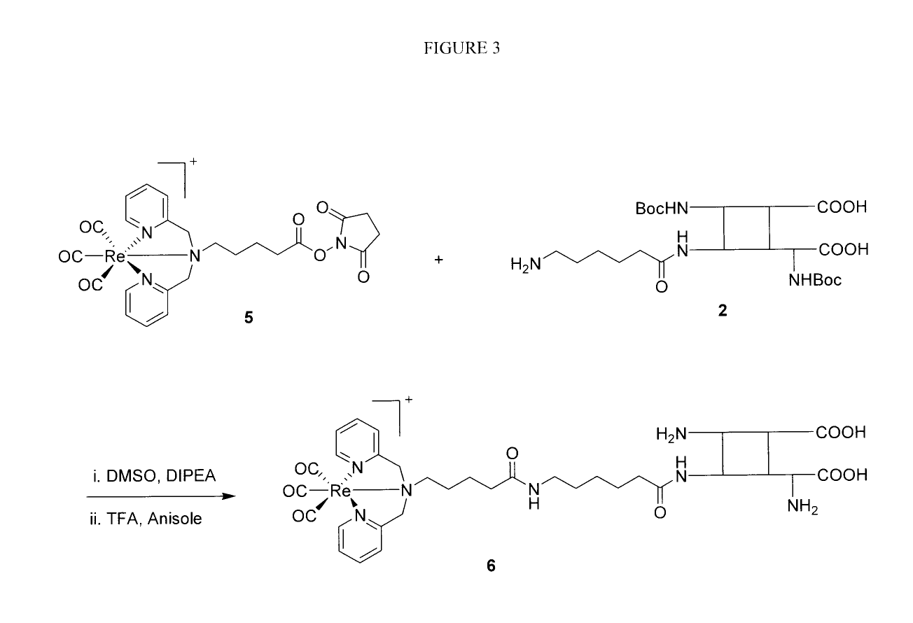

The bioconjugate, Re-BP-Pen-AHx-Insulin (6) was obtained by coupling the Boc-protected insulin precursor 2 with the active ester 5, in DMSO containing 5% N,N-diisopropylethylamine (DIPEA), followed by isolation by precipitation and centrifugation (FIG. 3). The protecting groups were removed using TFA containing 5% anisole and the desired material was isolated by preparative reversed-phase HPLC. The overall yield of Re-BP-Pen-AHx-Insulin was 46%, and the purity was greater than 95% as determined by HPLC (FIG. 2). Electrospray mass spectrometry was used to determine the identity of compound 6, which displayed a spectrum of multiply charged ions at m/z 2158.1 [M+3H⁺]/3, 1618.8 [M+4H⁺]/4, and 1295.2 [M+5H⁺]/5, which corresponded to the calculated molecular mass of the parent (6471.3 g/mol).

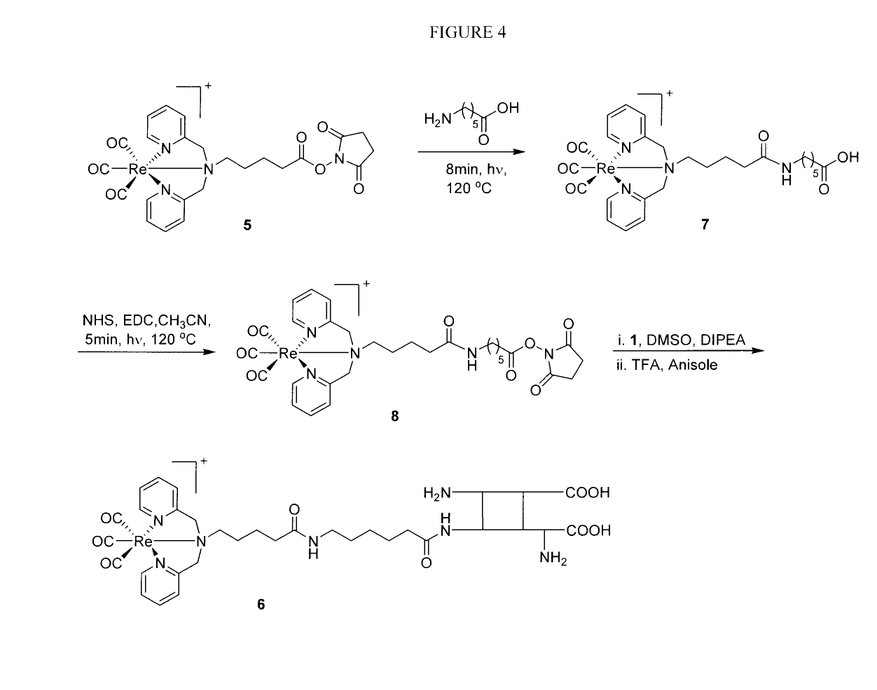

A more convergent synthetic route to 6 was developed subsequently, wherein diBoc-insulin 1 was conjugated to compound 8, which possesses the chelate and the desired aminohexanoic acid linker (FIG. 4). The active ester 8 was prepared from acid 7, which in turn was obtained by the reaction of 5 with commercially available 6-aminocaproic acid. While the overall yields were comparable in both approaches, the convergent route was found to be more convenient as it had fewer steps that required purification by preparative HPLC.

To verify the precise site of derivatization, digested fragments of compound 6 were analyzed by LC-MS. The sample was first treated with dithiothreitol to disrupt the interchain disulfide bonds, yielding separate A and B-chains. LC-MS (ESI(+)) analysis, for which the data is listed in Table 1, had three major peaks at 89.2, 98.0 and 105.9 minutes. The peak at 89.2 minutes exhibited a m/z value of 1190.6, which corresponds to the free unmodified A-chain. The peak at 98.0 minutes displayed m/z values of 1618.9 and 1295.3 corresponding to the [M+4H⁺]/4 and [M+5H⁺]/5 ions of the intact insulin bioconjugate. The final peak at 105.9 minutes had m/z values of 1024.0 and 1364.9, which corresponded to the [M+4H⁺]/4 and [M+3H⁺]/3 ions for the modified B-chain. These results confirm that the rhenium pendant group modification was not attached at the glycine-A1 site.

TABLE 1
LC-MS (ESI+) data for 6 following treatment with dithiothrietol (DTT).

| HPLC t_{R} ^{(a)} | m/z | Molecular | |
|---|---|---|---|
| (min) | found (calc.) | Ion | Fragment |
| 89.2 | 1190.6 (1192.9) | [M + 2H⁺]/2 | A-Chain |
| 98.0 | 1618.9 (1618.8) | [M + 4H⁺]/4 | Intact 6 |
| | 1295.3 (1295.2) | [M + 5H⁺]/5 | |
| 105.9 | 1364.9 (1365.9) | [M + 3H⁺]/3 | modified B-chain of 6 |
| | 1024.0 (1024.7) | [M + 4H⁺]/4 | |

^{(a)} Column C₁₈ Beckman Ultrasphere (150 × 4.6 mm, 5 μm particle). HPLC elution conditions - mobile phase
A: water with 0.1% TFA, mobile phase
B: acetonitrile with 0.05% TFA; gradient 99:1 (A/B) for 10 minutes. 99:1 (A/B) to 11:89 (A/B) over 140 minutes, 11:89 (A/B) to 1:99 (A/B) for 3 minutes, 1:99 (A/B) for 17 minutes: flow rate 0.1 mL/min. ESI + MS scanned from m/z 0−2400.

To verify the B-chain modification site, the sample was treated with endoproteinase Glu-C, which cleaves peptides at the carbonyl side of glutamic acid residues. LC-MS analysis of the peptide digest was performed and the results are summarized in Table 2. A peak at 66.7 minutes gave rise to a signal at m/z 417.4, which corresponds to the GIVE [M+H]⁺ fragment from the A-chain of insulin. A peak at 78.9 produced a signal at m/z 1116.7, which was consistent with the molecular ion [M+H]⁺ of the RGFFYTPKT fragment of the B-Chain. A peak at 95.5 minutes exhibited a m/z of 1129.4, which corresponded to the TFA adduct of the N-terminally modified FVNQHLCGSHLVE B-chain fragment [M+TFA+2H⁺]/2. Together, these data confirm that the site of conjugation was at the amino-terminus of the Phe-B1 site, and not at either the Lys-B29 or Gly-A1 primary amines.

TABLE 2
LC-MS (ESI+) data of DTT treated 6 digested
with DTT and endoproteinase Glu-C.

| HPLC t_{R} ^{(a)} | m/z | Molecular | |
|---|---|---|---|
| (min) | found (calc.) | Ion | Fragment |
| 66.7 | 417.4 (417.4) | [M + H⁺] | GIVE (A-Chain) |
| 78.9 | 1116.7 (1116.3) | [M + H⁺] | RGFFYTPKT (B-chain) |
| 95.5 | 1129.4 (1129.3) | [M + TFA + | Re-BP-Ahx- |
| | | 2H⁺]/2 | FVNQHLCGSHLVE |
| | | | (B-chain) |

^{(a)} Column C₁₈ Beckman Ultrasphere (150 × 4.6 mm, 5 μm particle). HPLC elution conditions - mobile phase
A: water with 0.1% TFA, mobile phase
B: acetonitrile with 0.05% TFA; gradient 99:1 (A/B) for 10 minutes. 99:1 (A/B) to 11:89 (A/B) over 140 minutes. 11:89 (A/B) to 1:99 (A/B) for 3 minutes. 1:99 (A/B) for 17 minutes; flow rate 0.1 mL/min. ESI + MS scanned from m/z 0−2400.

### Radiochemistry

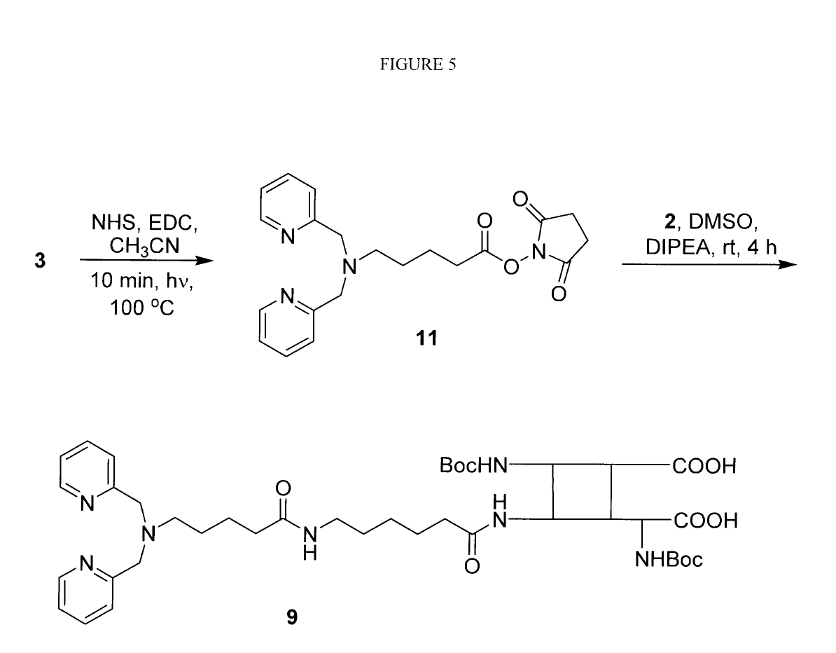

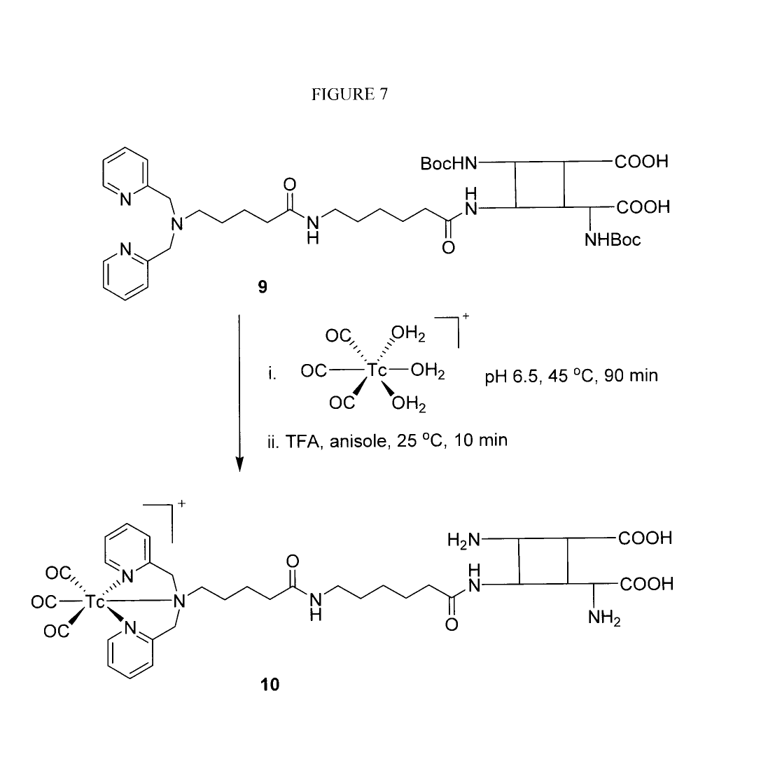

Production of ^{99m}Tc-based radiopharmaceuticals is typically performed using instant kits. As a result, the preparation of the ^{99m}Tc-analogue corresponding to 6 followed a direct labeling strategy suitable for use in an instant kit formulation. Compound 9, prepared in an analogous manner to its rhenium standard, (FIG. 5, 6) was selected as an appropriate precursor as it requires only one additional synthetic step (i.e. deprotection) to form the desired ^{99m}Tc-BP-Pen-AHx-insulin product (10) following addition of the [^{99m}Tc(CO)₃]⁺ core (FIG. 7). In keeping the Boc protecting groups present on the insulin precursor, the likelihood of non-selective coordination of the metal-core to the relatively nucleophilic primary amines on GlyA1 and LysB29 is reduced.

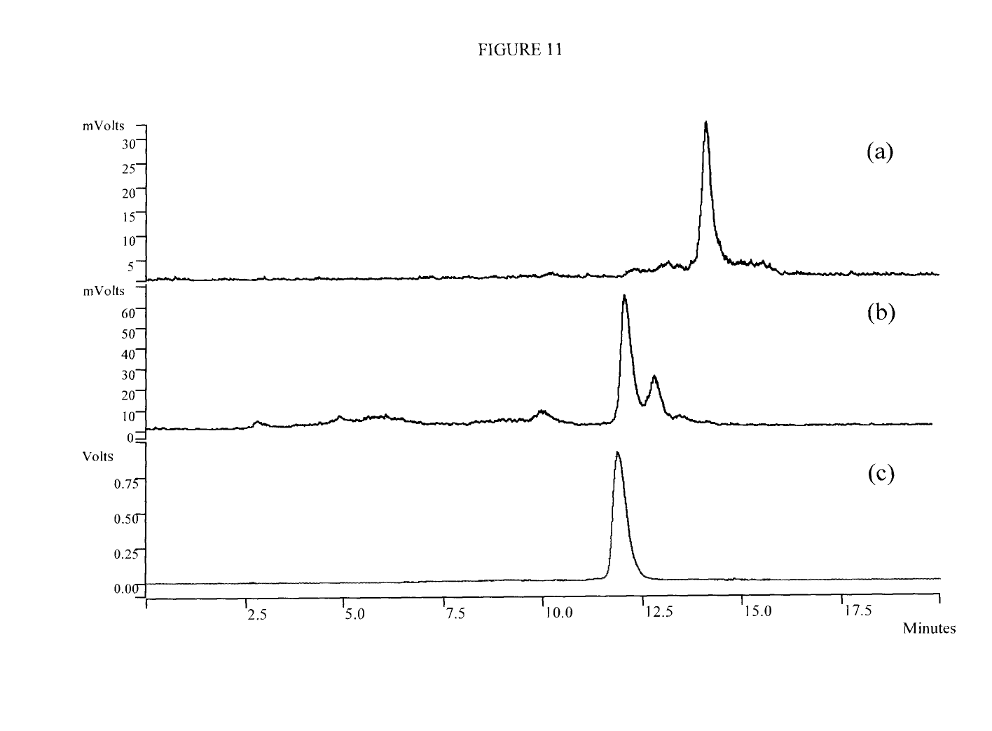

The technetium precursor, [^{99m}Tc(CO)₃(OH₂)₃]⁺, was formed from ^{99m}TcO₄⁺ using previously reported microwave methodology (Causey et al. Inorg. Chem. 2008, 47, 8213-8221). After cooling to room temperature, the pH of the solution containing the [^{99m}Tc(CO)₃(OH₂)₃]⁺ was varied between 5.0 and 7.5 using HCl. Compound 9 (2 mg, 312 nmol) was added in a mixture of CH₃CN and H₂O (1:2) and the solution stirred at various temperatures and times, and the reaction progress monitored by HPLC. The pH of the reaction mixture was found to be an important factor in determining the efficiency of labeling. The optimal pH identified was 6.5, which minimized the amount of non-specific labeling. This is likely due to the protonation of the histidine residues on insulin, which are known to be good donors for Tc(I). A combination of pH 6.5, a temperature of 45° C., and a reaction time of 90 minutes minimized the amount of non-specific labeling (FIG. 11a), and ultimately proved to be the most effective of the labeling conditions tested.

Following labeling, the reaction mixture was evaporated to dryness at 38° C. using a Biotage V10 solvent evaporator and then the Boc-groups were cleaved by dissolution of the dried mixture in TFA containing 5% anisole. The deprotected mixture was then purified by semi-preparative reverse-phase HPLC. The major product was collected, dried using the V10 evaporator, and resuspended in buffered saline containing 1% (w/w) bovine serum albumin (BSA). The final purified product 10 was obtained after a total reaction time of 4 hours in excellent radiochemical purity and 30% decay corrected yield (30±11%, n=4), and its retention time corresponded to the elution of the reference standard 6. This labeling strategy was amenable to producing sufficient quantities of labeled product (407 MBq; 11 mCi) from modest amounts of ^{99m}TcO₄⁻ (1.4 GBq; 38 mCi) for preclinical studies. Larger scale production runs have also been successfully completed using 11.1 GBq (300 mCi) of ^{99m}TcO₄⁻ generating 1.78 GBq (48 mCi; non-decay corrected) of 10 requiring slightly over one half-life (7 hrs) in which to isolate and reconstitute the product.

### Conclusions

A regioselective method for labeling insulin with ^{99m}Tc has been developed. The radioactive product was characterized by comparison to the non-radioactive and fully characterized reference standard 6. In a series of screening assays, the rhenium analogue retained the biological characteristics of native insulin, which supports the use of the ^{99m}Tc analogue as a tracer for studying insulin biodistribution and biochemistry in vivo.

### Methods, Materials and Instrumentation.

Reagents and solvents were purchased from Aldrich Inc., NovaBiochem Inc., or Fluka Inc. and were used without further purification. Human insulin was obtained from Aventis Inc and ¹²⁵I-insulin was obtained from Amersham Inc. Size-exclusion chromatography (SEC) was performed using HiTrap desalting cartridges (GE Healthcare). SEC cartridges were activated with 100 mM NH₄HCO₃ (20 mL) prior to use. Following the desalting, the cartridges were washed with the NH₄HCO₃ buffer (20 mL), H₂O (20 mL), and 80/20 (v/v) H₂O/EtOH (20 mL). Solid-phase extraction C18 SepPak cartridges (Waters) were activated with EtOH (10 mL) followed by H₂O (10 mL).

Both non-radioactive and radioactive analytical HPLC experiments were performed using Varian ProStar HPLC systems, fitted with a 330 PDA multiwavelength detector, a 230 solvent delivery module, and a Beckman Ultrasphere® C₁₈ column (4.6×100 mm, 300 Å, 5 μm) or Phenomenex Gemini® C₁₈ column (4.6×150 mm, 300 Å, 5 μm). For analytical experiments, mobile phases were A: H₂O+0.1% TFA and B: CH₃CN+0.05% TFA, and a gradient profile of 75/25 to 20/80 A/B (v/v) over 20 min, 20/80 A/B to 0/100 A/B over 5 min, followed by an isocratic wash of 0/100 A/B over 5 min (Method A). Absorbance data was collected from 210 to 400 nm where a wavelength of 254 nm was used to monitor the elution profiles.

For preparative and semi-preparative HPLC experiments, a Varian ProStar preparative HPLC system, which consisted of a model 320 detector, a model 215 solvent delivery module, and a Microsorb Dynamax® C₁₈ column (41.4×250 mm, 300 Å, 8 μm) for preparative experiments, and a Phenomenex Gemini® C₁₈ column (9.2×500 mm, 300 Å, 5 μm) for semi-preparative experiments were used. All purification runs were performed using H₂O+0.1% TFA (mobile phase A), and CH₃CN+0.05% TFA (mobile phase B). Absorbance was monitored at a wavelength of 254 nm. For purification of 1 and 2, the preparative C18 column was used and the elution protocol consisted of a gradient profile of 75/25 to 30/70 A/B (v/v) over 30 min, 30/70 A/B to 0/100 A/B over 5 min, followed by an isocratic wash of 0/100 A/B for 5 min, all at a flow rate of 45 mL/min (Method B). For purification of 6, 9 and 10, the semi-preparative C₁₈ column was used and the elution protocol consisted of a gradient profile of 75/25 to 20/80 A/B (v/v) over 20 min, 20/80 A/B to 0/100 A/B over 5 min, followed by an isocratic wash of 0/100 A/B over 5 min, all at a flow rate of 4 mL/min (Method C).

ESI-MS experiments and MALDI-MS experiments were carried out on Micromass Quattro Ultima and TOF Spec2E instruments, respectively. Prior to each MALDI TOF analysis, a calibration standard was run that consisted of a mixture of 1 fmol/μL substance P, 2 pmol/μL resin substrate tetradecapeptide, 2 pmol/μL adrenocorticotropic hormone fragment 18-39, and 10 pmol/μL cytochrome c. This was done in the positive ion reflection mode at 20 keV. NMR spectra were recorded using either a Bruker AV 600 MHz or DRX 500 MHz instrument. Chemical shifts (δ) are reported in ppm. Coupling constants (J) are reported in Hertz (Hz). NMR spectra were referenced to the residual proton peaks in the deuterated solvents (CHCl₃, 7.26 ppm; CH₃OH, 3.31 ppm) for ¹H NMR, and to the carbon signals of the deuterated solvents (CDCl₃, 77.16 ppm; CD₃OD, 49.0 ppm) for ¹³C NMR spectra.

### Synthetic Procedures

Bis(2-pyridylmethyl)pentanoic acid (BP-Pen) (3).

2-Pyridine carboxaldehyde (2.23 g, 20.8 mmol), 5-aminopentanoic acid (0.97 g, 8.3 mmol), and sodium triacetoxyborohydride (4.5 g, 21.3 mmol) were combined and stirred at room temperature in 30 mL of dichloroethane for 18 hrs, and then concentrated to dryness using a rotary evaporator. The residue was dissolved in 50 mL of water and extracted with 25 mL of ethyl acetate three times, and then the aqueous phase was concentrated to dryness using a rotary evaporator. The crude residue was purified by silica gel chromatography using 10/90 MeOH/CH₂Cl₂ (v/v) as the eluent. The product (1.5 g, 54%) was a yellow viscous liquid and its characterization data matched that reported in the literature.

### Re(CO)₃-BP-Pen (4).

A solution of [Re(CO)₃(OH₂)₃]Br (as described by Lazarova et al. Inorg. Chem. Comm. 2004, 7, 1023-1026) (1.04 g, 2.54 mmol) and 3 (462 mg, 1.54 mmol) in 3 mL of H₂O and 1 mL of CH₃CN were combined in a 2-5 mL Emery's process vial along with a magnetic stir bar and the reaction was crimp sealed. The sample was heated using a Biotage Initiator 60 microwave reactor at 150° C. for 5 minutes. The product was isolated by silica gel chromatography, and the separation was performed using 10/90 MeOH/CH₂Cl₂ (v/v) as the eluent. The product was an off-white solid (0.55 g, 62%) and its characterization data matched that reported in the literature.

### Succinimidyl [Re(CO)₃-BP-Pen] (5).

A solution of 4 (180 mg, 0.28 mmol), N-(3-dimethylaminopropyl)-N′-ethylcarbodiimide (EDC) (266 mg, 1.38 mmol) and N-hydroxy succinimide (NHS) (160 mg, 1.39 mmol) in 5 mL of acetonitrile were combined in a 2-5 mL Emery's process vial along with a magnetic stir bar and the vial was crimp sealed. The sample was heated using a Biotage Initiator 60 microwave reactor at 120° C. for 5 minutes. The crude reaction mixture was concentrated using a rotary evaporator, dissolved in CH₂Cl₂, extracted with water and the organic phase was concentrated to dryness. The crude sample was then purified using a Biotage SP1 purification system affixed with a disposable silica column, and separation performed using a solvent gradient 3/97 (v/v) MeOH/CH₂Cl₂ to 20/80 MeOH/CH₂Cl₂ (v/v). The product was isolated as an orange oil (192 mg, 91%) and its characterization data matched that reported in the literature.

### Re(CO)₃-BP-Pen-AHx (7).

Method A: A solution of 5 (167 mg, 0.25 mmol) in 5 mL of CH₃CN and 6-aminocaproic acid (170 mg, 1.30 mmol) were combined in a 2-5 mL Emery's process vial along with a magnetic stir bar and the vial crimp sealed. The sample was heated using a Biotage Initiator 60 microwave reactor at 120° C. for 8 minutes. The precipitate (unreacted aminocaproic acid) was removed by filtration and the crude reaction mixture was concentrated using a rotary evaporator. The sample was then purified using silica gel chromatography and a solvent gradient 5/95 (v/v) MeOH/CH₂Cl₂ to 20/80 MeOH/CH₂Cl₂ (v/v). The product was isolated as an off-white solid (104 mg, 61%).

Method B: To a solution of 12 (164 mg, 0.4 mmol) in 3.8 mL water and 1.2 mL acetonitrile in a 2-5 mL Emery's process containing a magnetic stir bar, was added [Re(CO)₃(OH₂)₃]Br (279 mg, 0.69 mmol) and the vial crimp sealed. The sample was heated using a Biotage Initiator 60 microwave reactor at 150° C. for 5 minutes and the solution concentrated to dryness using a rotary evaporator. The residue was then purified using silica gel chromatography and separation was performed using a solvent gradient 10/90 (v/v) MeOH/CH₂Cl₂ to 20/80 MeOH/CH₂Cl₂ (v/v). The product was obtained as an off-white solid (265 mg, 98%). m.p. 103° C. R_{f} (CH₂Cl₂/MeOH: 90:10, v/v): 0.35. High resolution ES MS (+) calcd for C₂₆H₃₂O₆N₄Re, m/z 681.1841 and 683.1880 for ¹⁸⁵Re and ¹⁸⁷Re. found 681.1802 and 683.1841 respectively. ¹H NMR δ (500.13 MHz, CD₃OD): 8.86 (d, J=5.5, 2H, PyH), 7.94 (t, J=7.8, 2H, PyH), 7.58 (d, J=8.0, 2H, PyH), 7.37 (t, J=6.5, 2H, PyH), 4.87 (dd, J=16.5 and 23.0, 4H, PyCH₂), 3.83 (m, 2H, NCH₂), 3.21 (t, J=7.0, 2H, CONHCH₂), 2.35 (t, J=7.5, 2H, CH₂COOH), 2.31 (t, J=7.5, 2H, CH₂CO), 1.98 (m, 2H, CH₂), 1.75 (m, 2H, CH₂), 1.64 (m, 2H, CH₂), 1.55 (m, 2H, CH₂), 1.40 (m, 2H, CH₂). ¹³C NMR δ (125.76 MHz, CD₃OD): 197.2, 196.4, 177.4, 175.3, 162.2, 153.1, 141.6, 126.9, 124.7, 71.5, 68.8, 40.3, 36.3, 34.9, 30.1, 27.5, 25.7, 24.0.

### Succinimidyl [Re(CO)₃-BP-Pen-AHx] (8).

A solution containing 7 (240 mg, 0.35 mmol), EDC (337 mg, 1.76 mmol) and NHS (202 mg, 1.76 mmol) in 4 mL of acetonitrile was placed in a 2-5 mL Emery's process vial along with a magnetic stir bar and the vial crimp sealed. The sample was heated using a Biotage Initiator 60 microwave reactor for 5 minutes at 120° C. The crude reaction mixture was concentrated using a rotary evaporator, the residue dissolved into CH₂Cl₂ which was then extracted with water and the organic phase concentrated to dryness. The sample was then purified by silica gel chromatography using 10/90 MeOH/CH₂Cl₂ (v/v). The product was isolated as a pale yellow oil (245 mg, 89%). R_{f} (CH₂Cl₂/MeOH: 90:10, v/v): 0.48. High resolution ES MS (+) calcd for C₃₀H₃₅O₈N₅Re, m/z 778.2038 and 780.2043 for ¹⁸⁵Re and ¹⁸⁷Re. found 778.2029 and 780.2036 respectively. ¹H NMR δ (500.13 MHz, CD₃OD): 8.85 (d, J=5.0, 2H, PyH), 7.94 (t, J=8.0, 2H, PyH), 7.57 (d, J=8.0, 211, PyH), 7.37 (t, J=6.5, 2H, PyH), 4.86 (dd, J=16.5 and 12.0, 4H, PyCH₂′), 3.83 (m, 214, NCH₂), 3.22 (t, J=7.0, 2H, CONHCH₂), 2.83 (s, 4H succinimidyl-CH₂), 2.65 (m, 2H, CH₂CO), 2.36 (m, 2H, CH₂), 1.98 (m, 2H, CH₂), 1.76 (m, 4H, CH₂), 1.53 (m, 4H, CH₂). ¹³C NMR δ (125.76 MHz, CD₃OD): 197.2, 175.3, 171.8, 170.2, 162.1, 153.1, 141.6, 126.9, 124.6, 71.4, 68.8, 54.8, 40.1, 36.3, 31.5, 29.8, 27.0, 26.5, 26.3, 25.8.

### Re-BP-Pen-AHx-Insulin (6)

Step 1: Method A: A solution of B¹-(6-aminohexanoyl)-A¹,B²⁹-di-(tert-butyloxycarbonyl)insulin (AHx-DBI, 2) (10.0 mg, 1.3 μmol) and 5 (12.2 mg, 16.3 μmol) in DMSO (1.25 mL) containing N,N-diisopropylethylamine (DIPEA) (25 μL) was stirred for 90 minutes at room temperature. The reaction mixture was then transferred to a centrifuge vial containing 15 mL of CH₃CN then Et₂O was added slowly until a white precipitate formed. The precipitate was isolated by centrifugation at 3500 rpm for 30 minutes at 5° C. The resulting pellet was washed twice with 100% CH₃CN and re-isolated by centrifugation. The pellet was then dissolved in 75/25 H₂O/CH₃CN containing 0.1% TFA and purified by preparative reverse-phase HPLC. The desired fractions were collected and concentrated by rotary evaporation (water bath 37° C.) to remove the majority of the CH₃CN. Following lyophilization, Re-BP-Pen-AHx-DBI (7.1 mg, 65%) was obtained as a white powder. Analytical HPLC t_{R}: 9.8 minutes (Beckman Ultrasphere C₁₈ column 100×4.6 mm, method A). ES MS (+) calcd 1335.2 [M+5H⁺]/5, 1668.8 [M+4H⁺]/4. found 1335.4 and 1668.9 respectively.

Method B: A solution of A¹,B²⁹-di-(tert-butyloxycarbonyl)insulin (DBI, 1) (10.0 mg, 1.67 μmol) and 8 (13 mg, 16.7 μmol) in DMSO (0.4 mL) containing DIPEA (5%) was stirred for 2 hours at room temperature. The reaction mixture was then transferred to a centrifuge vial containing 15 mL of CH₃CN and Et₂O was added slowly until a white precipitate formed. The precipitate was isolated by centrifugation at 3500 rpm for 30 minutes at 5° C., then washed twice with 100% CH₃CN and isolated by centrifugation. The purification procedure was the same as in method A. Following lyophilization, Re-BP-Pen-AHx-DBI (4.2 mg, 38%) was obtained as a white powder.

Step 2: Re-BP-Pen-AHx-DBI (5.0 mg) was dissolved in 500 μL of TFA containing 5% anisole (v/v) and allowed to react at room temperature for 30 minutes. The deprotected product was then precipitated in 25 mL of Et₂O and isolated by centrifugation at 3500 rpm for 30 minutes at 5° C. The precipitate was washed twice with 100% CH₃CN followed by centrifugation. The solid was then dissolved in 75/25 H₂O/CH₃CN containing 0.1% TFA and purified by preparative reverse-phase HPLC. The desired fractions were collected and concentrated by rotary evaporation (water bath 37° C.) to remove the majority of the CH₃CN. Following lyophilization, Re-BP-Pen-AHx-Insulin (3.4 mg, 71%) was obtained as a white powder. Analytical HPLC (method A) t_{R}=9.2 min (Phenomenex Gemini C₁₈ 150×4.6 mm). ES MS (+) calcd 1079.5 [M+6H⁺]/6, 1295.2 [M+5H⁺]/5, 1618.8 [M+4H⁺]/4, 2158.0 [M+3H⁺]/3. found 1079.7, 1295.1, 1618.8, 2158.8.

### Succinimidyl BP-Pen (11).

Bis(2-pyridylmethyl)pentanoic acid 3 (0.40 g, 1.3 mmol), EDC (1.04 g, 6.7 mmol), and NHS (0.77 g, 6.7 mmol) and 5 mL of acetonitrile were combined in a 2-5 mL Emery's process vial along with a magnetic stir bar and the container crimp sealed. The sample was heated using a Biotage Initiator 60 microwave reactor at 100° C. for 10 minutes. The crude reaction mixture was concentrated using a rotary evaporator, the residue dissolved into CH₂Cl₂ which was then extracted with water and the organic phase was concentrated to dryness leaving a viscous orange liquid. The residue was then dissolved in MeOH, filtered, concentrated to dryness, the residue dissolved in a minimum volume of CH₂Cl₂ and the desired product isolated by silica chromatography using a Biotage SP1 purification system and a solvent gradient of 3/97 (v/v) MeOH/CH₂Cl₂ to 20/80 MeOH/CH₂Cl₂ (v/v). The product (0.41 g, 77%), was an orange viscous liquid. R_{f} (CH₂Cl₂/MeOH: 90:10, v/v): 0.65. High resolution ES MS (+) calcd for C₂₁H₂₅O₄N₄ (M+H⁺) m/z 397.1876. found 397.1872. ¹H NMR δ (500.13 MHz, CD₃OD): 8.44 (d, J=4.5 Hz, 2H, PyH), 7.79 (m, 2H, PyH), 7.62 (d, J=8.0, 2H, PyH), 7.28 (t, J=6.0, 2H, PyH), 3.84 (s, 4H, PyCH₂), 2.67 (s, 4H, succinimidyl CH₂), 2.56 (m, 2H, CH₂N), 2.23 (d, J=7.0, 21-1, CH₂CO₂N), 1.57 (m, 4H, CH₂). ¹³C NMR δ (125.76 MHz, CD₃OD): 175.7, 174.9, 160.6, 149.4, 138.6, 124.9, 123.7, 61.1, 55.1, 51.9, 34.3, 27.4, 26.3, 23.6.

### BP-Pen-AHx (12).

Compound 11 (250 mg, 0.63 mmol), was combined with 6-aminocaproic acid (496 mg, 3.78 mmol) and 5 mL acetonitrile in a 2-5 mL Emery's process vial along with a magnetic stir bar and the container crimp sealed. The mixture was heated in a Biotage Initiator 60 microwave reactor at 120° C. for 8 minutes. The precipitate (unreacted aminocaproic acid) was removed by filtration, and the filtrate was evaporated to give a pale yellow residue. Purification by silica gel chromatography using a solvent gradient MeOH/CH₂Cl₂ 10:90 to 15:85 (v/v) as the eluent gave a pale yellow oil (196 mg, 75%). R_{f} (CH₂Cl₂/MeOH: 90:10, v/v): 0.40. High resolution ES MS (+) calcd for C₂₃H₃₃O₃N₄ (M+H⁺) m/z 413.2553. found 413.2571. ¹H NMR δ (500.13 MHz, CD₃OD): 8.45 (d, J=5.0, 2H, PyH), 7.80 (t, J=7.5, 2H, PyH), 7.61 (d, J=7.5, 21-1, PyH), 7.29 (m, 2H, PyH), 3.87 (s, 4H, PyCH₂), 3.14 (t, J=7.0, 2H, NHCH₂), 2.63 (t, J=6.0, 2H, NCH₂), 2.27 (t, J=7.5, 2H, CH₂CO₂H), 2.12 (m, 2H, CH₂CONH), 1.60 (m, 6H, CH₂), 1.50 (m, 2H, CH₂), 1.36 (m, 2H, CH₂). ¹³C NMR δ (125.76 MHz, CD₃OD): 177.8, 175.8, 159.8, 149.5, 138.7, 124.9, 123.9, 60.9, 55.5, 40.2, 36.7, 35.1, 30.1, 27.6, 27.3, 25.8, 24.7, 21.0.

### Succinimidyl BP-Pen-AHx (13).

Compound 12 (100 mg, 0.24 mmol), EDC (230 mg, 1.2 mmol), and NHS (138 mg, 1.2 mmol) in 3 mL of acetonitrile were combined in a 2-5 mL Emery's process vial along with a magnetic stir bar and the container crimp sealed. The sample was heated using a Biotage Initiator 60 microwave reactor at 120° C. for 10 minutes. The crude reaction mixture was concentrated using a rotary evaporator, dissolved in CH₂Cl₂ extracted with water, and the organic phase was concentrated to give a dark yellow oil. The residue was then dissolved in CH₂Cl₂ and purified by silica gel chromatography using 10/90 MeOH/CH₂Cl₂ (v/v) as the eluent. The product (110 mg, 89%), was isolated as a yellow oil. R_{f} (CH₂Cl₂/MeOH: 90:10, v/v): 0.50. High resolution ES MS (+) calcd for C₂₇H₃₆O₅N₅ (M+H⁺) m/z 510.2716. found 510.2721. ¹H NMR δ (500.13 MHz, CD₃OD): 8.43 (d, J=4.5, 2H, PyH), 7.79 (t, J=7.5, 2H, PyH), 7.62 (d, J=8.0, 2H, PyH), 7.27 (t, J=6.0, 2H, PyH), 3.81 (s, 4H, PyCH₂), 3.15 (t, J=6.7, 2H, CH₂NHCO), 2.82 (s, 4H, succinimidyl CH₂), 2.61 (t, J=7.2, 2H, CH₂N), 2.56 (m, CH₂CO₂N), 2.12 (m, 2H, CH₂CONH), 1.73 (m, 2H, CH₂), 1.55 (m, CH₂), 1.44 (m, 2H, CH₂). ¹³C NMR δ (125.76 MHz, CD₃OD): 175.9, 171.8, 170.2, 160.6, 149.4, 138.6, 124.9, 123.8, 61.1, 55.4, 40.0, 36.8, 31.5, 29.8, 27.6, 27.0, 26.5, 25.3, 24.8.

### BP-Pen-AHx-DBI (9)

Method A: A solution of 2 (83 mg, 13 μmol) and 11 (30 mg, 76 μmol) in DMSO (1.25 mL) containing DIPEA (25 μL) was stirred for 4 hrs at room temperature. The reaction mixture was then transferred to a centrifuge vial containing 15 mL of CH₃CN and Et₂O added slowly until a white precipitate formed. The precipitate was isolated by centrifugation at 3500 rpm for 30 minutes at 5° C., and the resulting pellet washed twice with 100% CH₃CN and re-isolated by centrifugation. The pellet was then dissolved in 75/25 H₂O/CH₃CN containing 0.1% TFA and the desired product was isolated by preparative reverse-phase HPLC. The fractions containing 9 were collected and concentrated by rotary evaporation (37° C.) to remove the majority of the CH₃CN. Following lyophilization, BP-Pen-AHx-DBI (53 mg, 64%) was obtained as a white powder. Analytical HPLC (Phenomenex Gemini C₁₈ column 100×4.6 mm, method A) t_{R}=9.03 min. MALDI TOF MS (+) m/z calcd 6403 [M+H⁺]. found, 6403.1; ES MS (+) calcd 1285.2 [M+4H⁺+NH₄⁺]/5, 1601.5 [M++4H⁺]/4, 2135.0 [M+3H⁺]/3. found, 1285.2, 1601.4, 2134.8.

Method B: Coupling with DBI: A solution of 1 (41 mg, 6.8 μmol) and 13 (35 mg, 68.7 μmol) in DMSO (0.4 mL) containing DIPEA (5%) was stirred for 4 h at room temperature. The reaction mixture was then transferred to a centrifuge vial containing 15 mL of CH₃CN then Et₂O was added slowly until a white precipitate formed. The precipitate was isolated by centrifugation at 3500 rpm for 30 minutes at 5° C., then washed twice with 100% CH₃CN followed by centrifugation. The purification procedure was the same as in method A. Following lyophilization, BP-Pen-AHx-DBI (25 mg, 57%) was obtained as a white powder.

### Example 2

### In vitro Testing of Re-BP-AHx-Insulin

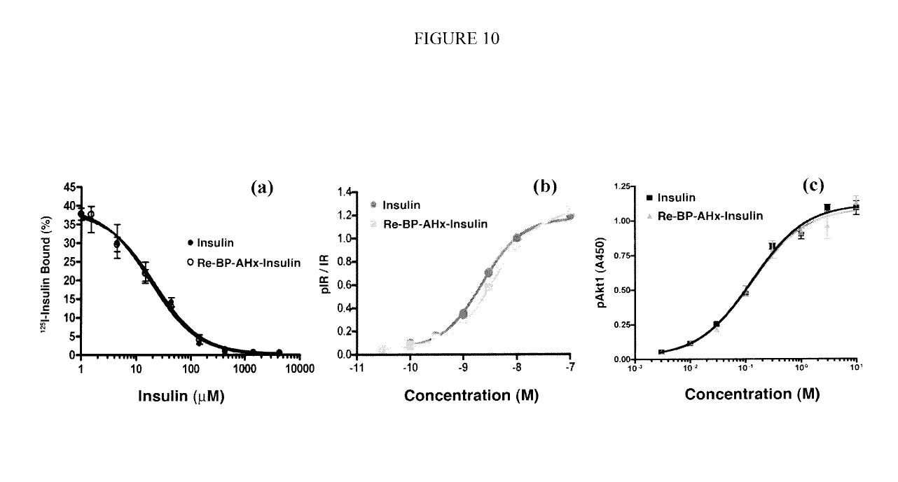

To evaluate the biological properties of compound 6, three in vitro assays were performed to probe the vital points within the insulin-signaling cascade. The first assay was performed to determine the ability of 6 to bind to the insulin receptor by assessing the displacement of ¹²⁵I-insulin from the IR by both 6 and native insulin. The behavior of 6 bound to the IR was found to be markedly similar to that of native insulin (FIG. 10a), giving IC₅₀ values of 17.8 nM and 11.7 nM, for the modified and native insulin, respectively. Although the binding properties were similar for the native and modified insulin, further evidence to confirm that 6 retained its core physiological functions was obtained by two functional in vitro assays. The insulin receptor autophosphorylation was assessed through the use of an enzyme-linked immunosorbant assay (ELISA). It was found that there was no significant difference observed in the extent of autophosphorylation induced by 6 compared to unmodified human insulin (FIG. 10b). The calculated EC₅₀ for autophosphorylation by unmodified human insulin was 2.0 nM, whereas that for Re-BP-Pen-AHx-insulin was 3.2 nM. Finally, to probe the downstream signaling resulting from insulin binding, a second ELISA experiment was performed to monitor stimulation of Akt phosphorylation (S473). It was found that the response to Re-BP-Pen-AHx-insulin binding was not significantly different from that induced by unmodified human insulin (FIG. 10c). The calculated EC₅₀ for stimulation of Akt1 phosphorylation was 0.13 nM for both human insulin and compound 6.

The results of the biochemical assays showed that chemical modification at the PheB1 site had minimal impact on the binding of insulin to the IR and supports the use of the iso-structural ^{99m}Tc-analogue as a mimic of insulin for in vivo studies.

### Methods

### Digestion Studies

Digestion studies were performed using a previously reported procedure for derivatized insulin (Guenther et al. J. Med. Chem. 2006, 49, 1466-1474). To 250 μL of compound 6 (2.5 mg/mL) in PBS, was added 25 μL of 0.4 M aqueous NH₄HCO₃ in a plastic conical vial, and the mixture was agitated gently. To this was added 5 μL of 45 mM aqueous dithiothreitol and the mixture incubated for 15 min at 50° C. After cooling to room temperature, 5 μL of 100 mM aqueous iodoacetamide was added and the solution agitated periodically over 15 min. An aliquot (56 μL) was mixed with 28 μL of 0.4 M NH₄HCO₃ in 8M aqueous urea and the analysis performed by LCMS (ES+).

The remaining solution from the dithiothreitol experiment was transferred to a glass vial. To this was added 2.5 μL of endoproteinase-Glu-C in water, and the mixture incubated for 16 h at 37° C. An aliquot (56 μL) was mixed with 28 μL of 0.4 M NH₄HCO₃ in 8M aqueous urea and the analysis performed by LCMS (ES+).

### ¹²⁵I-Insulin Competitive Binding Assay:

Human embryonic kidney (HEK) cell lines were stably transfected to allow expression of a large number of human insulin receptors (hIR-293 cells). The hIR-293 cells were incubated in the presence of 140 pM ¹²⁵I-insulin (Amersham) and varying concentrations of either unlabeled recombinant human insulin (Aventis, lot A0136-1) or Re-BP-AHx-insulin for 120 minutes at 4° C. and then washed three times. The cell pellets were counted on a gamma counter and reported as percent binding (CPM sample/CPM added). The concentration of the insulin for a 50% displacement was also calculated using non-linear regression analysis (Prism version 4.0).

### Insulin Receptor Autophosphorylation Assay:

Chinese Hamster ovary cells were stably transfected to express a large number of human insulin receptors (CHO-hIR cells). The CHO-hIR cells were serum deprived for 1 h, then incubated with either human insulin (Aventis) or Re-BP-AHx-insulin at various concentrations for 10 minutes. Cells were then lysed, and the lysates were clarified by centrifugation and applied to 96 well plates coated with anti-insulin receptor monoclonal antibodies. The extent of autophosphorylation was determined by quantitation of the binding of a second antibody directed against phosphotyrosine residues (HRP-conjugated PY20, Oncogene Research Products) using a coupled horseradish peroxidase reaction (Enzyme-linked Immunosorbent Assay (ELISA)), and measurements were obtained by monitoring the colorimetric reaction using a UV spectrophotometer.

### Akt1 Activation:

Confluent H4IIE cells were serum deprived for 4 hours in serum containing β-mercaptoethanol and 0.25% bovine serum albumin. Triplicate wells were treated with human insulin (Aventis) or Re-BP-AHx-insulin at various concentrations for 10 minutes. Cells were lysed in 0.5 ml 1× Cell Lysis Buffer (Cell Signaling, #7160) and cells broken open by brief sonication. Lysates were collected and centrifuged for 10 minutes at 14,000 rpm. Supernatants were recovered, diluted 1:1 with Sample Diluent, and phospho-Akt1 was quantitated in 100 μL of each diluted supernatant using the PathScan phospho-Akt1 (Ser473) ELISA (Cell Signaling, #7160).

### Radiochemistry

Caution: ^{99m}Tc is radioactive and should only be handled in a licensed facility using the appropriate shielding.

B¹-[^{99m}Tc(CO)₃{bis(2-pyridylmethyl)pentanoyl-6-aminohexanoyl)}]insulin (^{99m}Tc-BP-Pen-AHx-Insulin, 10) Boranocarbonate (8.5 mg), K⁺/Na⁺ tartrate (15 mg), NaB₄O₇.10H₂O (3 mg), and Na₂CO₃ (4 mg), were added to 2-5 mL Emery's process vial along with a magnetic stir bar. The vial was crimp sealed and purged with argon. Approximately 1 mL of ^{99m}TcO₄⁻ in saline from a ⁹⁹Mo/^{99m}Tc generator (1.11-1.85 GBq; 30-50 mCi) was added and the sample was heated at 130° C. in a microwave reactor for 3 minutes. The solution containing [^{99m}Tc(CO)₃(OH₂)₃]⁺ was cooled to room temperature and 2.5 N HCl was carefully added to bring the pH of the solution to 6.5. Compound 9 (2 mg, 312 nmol) was dissolved in 30/60 (v/v) CH₃CN/H₂O (0.25 mL) and added by syringe to the solution of [^{99m}Tc(CO)₃(OH₂)₃]⁺ and the reaction was heated in an aluminium heating block at 45° C. for 90 min. The crude reaction mixture was then transferred to a 20 mL scintillation vial and concentrated to dryness using a Biotage V10 evaporator. To the dried sample, 700 μL of TFA containing 5% (v/v) anisole was added and the sample was dissolved using the re-dissolve mode on the V10 evaporator. After 10 minutes the majority of the TFA was removed using the evaporator and the sample dissolved in a solution of 25/75 (v/v) CH₃CN/H₂O containing 0.1% TFA, which was injected into an equilibrated semi-preparative reversed-phase C18 column (Phenomenex Gemini® 500×9.4 mm). The radioactive peak corresponding to the desired product was then collected into a 20 mL scintillation vial and concentrated to dryness using the V10 evaporator (T=37° C.). The final purified product was dissolved in sterile phosphate buffered saline containing 1% bovine serum albumin (BSA) and filtered through a 0.2 μm filter (d.c.y. 30±11%, n=4).

1. A radiolabelled insulin analogue, comprising a radiolabel linked to an insulin analogue at an amino acid at the terminal end of the B chain of the insulin analogue.

2. An analogue as defined in claim 1, wherein the radiolabel is linked to an amino acid at the N-terminal end of the B-chain.

3. An analogue as defined in claim 2, wherein the radiolabel is linked to the N-terminus of the B-chain.

4. An analogue as defined in claim 1, wherein the insulin analogue is mammalian insulin.

5. An analogue as defined in claim 4, wherein the insulin analogue is human insulin.

6. An analogue as defined in claim 1, wherein the radiolabel is linked to the insulin analogue via a chelator.

7. An analogue as defined in claim 6, wherein the chelator is linked to the insulin analogue via a linker.

8. An analogue as defined in claim 1, wherein the radiolabel is selected from the group consisting of fluorine-18, gallium-67, krypton-81m, rubidium-82, technetium-99, indium-111, iodine-123, xenon-133 and thallium-201.

9. An analogue which is compound 10.

10. A method of preparing a radiolabelled insulin analogue as defined in claim 1 comprising the steps of:
i) linking a chelator to a terminal amino group on the B-chain of the insulin analogue; and
ii) reacting the linked chelator with a radioisotope under suitable conditions.

11. The method of claim 10, wherein the chelator is linked to an amino acid at the N-terminal end of the B-chain of the insulin analogue.

12. The method of claim 10, wherein the insulin analogue is mammalian insulin.

13. The method of claim 10, wherein the chelator includes a Bis(2-pyridylmethyl) group.

14. The method of claim 10, wherein the radioisotope is selected from the group consisting of fluorine-18, gallium-67, krypton-81m, rubidium-82, technetium-99, indium-111, iodine-123, xenon-133 and thallium-201.

15. The method of claim 10, wherein the conditions for linking the radioisotope include a pH in the range of about 6-6.8, a temperature in the range of about 40-50° C. and a reaction time of at least about 60 minutes.

16. A kit comprising a chelator-linked insulin analogue and a radioisotope to be reacted therewith.

17. A method of imaging a mammal comprising administering a radiolabelled insulin analogue as defined in claim 1 to the mammal.

18. The method of claim 17, wherein the mammal has an insulin-related disorder.
